(19) United States
(12) Patent Application Publication
(10) Pub. No.: US 2007/0259404 A1
(43) Pub. Date: Nov. 8, 2007

(54) Method of Producing Heterologous Proteases

(75) Inventors: Steen Troels Jorgensen, Allerod (DK); Niels Banke, Soborg (DK); Mogens Wumpelmann, Herlev (DK)

Correspondence Address: NOVOZYMES NORTH AMERICA, INC., 500 FIFTH AVENUE, SUITE 1600, NEW YORK, NY, 10110, US

(73) Assignee: NOVOZYMES A/S, Bagsvaerd (DK)

(21) Appl. No.: 11/630,314

(22) PCT Filed: Jun. 20, 2005
(86) PCT No.: PCT/DK05/00408

§ 371 Date: Dec. 20, 2006

Related U.S. Application Data
(60) Provisional application No. 60/581,836, filed on Jun. 21, 2004.

(30) Foreign Application Priority Data
Jun. 21, 2004 (DK) PA 2004 00973

(51) Int. Cl. C12P 1/00

(52) U.S. Cl. 435/71.1

(57) ABSTRACT

The present invention provides improved methods of producing S2A (or S1E) proteases in Gram-positive expression host cells, the method comprising the steps of (a) cultivating in a fed-batch fermentation a Gram-positive cell comprising at least one polynucleotide encoding the heterologous S2A/S1E protease under conditions conducive for production of the protease, wherein at least 20% of the duration of said cultivating takes place at a temperature of below 36.5OC; and (b) recovering the protease.

### FIELD OF INVENTION

A number of microbially derived related proteases are notably difficult to produce in industrially relevant yields, they may be prone to various types of degradation and/or instabilities. The present invention provides improved methods of producing S2A (or S1E) proteases in Gram-positive expression host cells.

### BACKGROUND

Polypeptides having protease activity, or proteases, are sometimes also designated peptidases, proteinases, peptide hydrolases, or proteolytic enzymes. Proteases may be of the exo-type that hydrolyses peptides starting at either end thereof, or of the endo-type that act internally in polypeptide chains (endopeptidases). Endopeptidases show activity on N— and C-terminally blocked peptide substrates that are relevant for the specificity of the protease in question.

A protease is an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmb/enzyme/index.html.

Proteases are classified on the basis of their catalytic mechanism into the following groups: Serine proteases (S), Cysteine proteases (C), Aspartic proteases (A), Metalloproteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

Serine proteases are ubiquitous, being found in viruses, bacteria and eukaryotes; they include exopeptidase, endopeptidase, oligopeptidase and omega-peptidase activity. Over 20 families (denoted S1-S27) of serine proteases have been identified, these being grouped into 6 clans denoted SA, SB, SC, SE, SF, and SG, on the basis of structural similarity and functional evidence (Barrett et al. 1998. Handbook of proteolytic enzymes). Structures are known for at least four of the clans (SA, SB, SC and SE), these appear to be totally unrelated, suggesting at least four evolutionary origins of serine peptidases. Alpha-lytic endopeptidases belong to the chymotrypisin (SA) clan, within which they have been assigned to subfamily A of the S2 family (S2A).

Another classification system of proteolytic enzymes is based on sequence information, and is therefore used more often in the art of molecular biology; it is described in Rawlings, N.D. et al., 2002, MEROPS: The protease database. Nucleic Acids Res. 30:343-346. The MEROPS database is freely available electronically at http://www.merops.ac.uk. According to the MEROPS system, the proteolytic enzymes classified as S2A in ‘The Handbook of Proteolytic Enzymes’, are in MEROPS classified as ‘S1E’ proteases (Rawlings N.D., Barrett A J. (1 993) Evolutionary families of peptidases, Biochem. J. 290:205-218).

A number of industrially interesting S2A/S1E proteases derived from various Nocardiopsis species are difficult to produce in significant yields by recombinant production in the preferred industrial Gram-positive expression host cells. Even incremental improvements in the production yields of these proteases are highly interesting for the enzyme industry. The present invention provides improved methods of producing S2A/S1E proteases in Gram-positive host cells resulting in higher yields.

### SUMMARY OF THE INVENTION

The present inventors found that lowering the fermentation temperature, either for the whole duration of the fermentation or in a part of the fermentation, below the usual 37° C. employed for industrial fermentations of Gram-positive microorganisms, resulted in significant yield increases.

Accordingly, in a first aspect, the present invention relates to a method of producing a heterologous S2A/S1E protease in a Gram-positive host cell, the method comprising the steps of:
(a) cultivating in a fed-batch fermentation a Gram-positive cell comprising at least one polynucleotide encoding the heterologous S2A/S1E protease under conditions conducive for production of the protease, wherein at least 20% of the duration of said cultivating takes place at a temperature of below 36.5° C.; and
(b) recovering the protease.
   Definitions

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”) DNA Cloning: A Practical Approach, Volumes I and II/D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

A “nucleic acid molecule” or “nucleotide sequence” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization.

For purposes of the present invention, hybridization indicates that the nucleotide sequence hybridizes to a labeled polynucleotide probe which hybridizes to the nucleotide sequences shown in SEQ ID NO's: 3, 5, 9, 13, 17, 19, 21, 23, 25, 27, 29, 31, 33, 35, 37, or 39 under very low to very high stringency conditions. Molecules to which the polynucleotide probe hybridizes under these conditions may be detected using X-ray film or by any other method known in the art. Whenever the term “polynucleotide probe” is used in the present context, it is to be understood that such a probe contains at least 15 nucleotides.

In an interesting embodiment, the polynucleotide probe is the complementary strand of a fragment of at least 15 nucleotides of one of SEQ ID NO's: 3, 5, 9, 13, 17, 19, 21, 23, 25, 27, 29, 31, 33, 35, 37, or 39. In another interesting embodiment, the polynucleotide probe is a fragment of at least 15 nucleotides of the complementary strand of any nucleotide sequence which encodes the polypeptide of SEQ ID NO's: 2, 12, 14, 16, 18, 20, 22, 24, or 26. In a further interesting embodiment, the polynucleotide probe is the complementary strand of SEQ ID NO's: 3, 5, 9, 13, 17, 19, 21, 23, 25, 27, 29, 31, 33, 35, 37, or 39. In a still further interesting embodiment, the polynucleotide probe is the complementary strand of the mature polypeptide coding region of SEQ ID NO's: 3, 5, 9, 13, 17, 19, 21, 23, 25, 27, 29, 31, 33, 35, 37, or 39.

For long probes of at least 100 nucleotides in length, very low to very high stringency conditions are defined as prehybridization and hybridization at 42° C. in 5X SSPE, 1.0% SDS, 5X Denhardt's solution, 100 microg/ml sheared and denatured salmon sperm DNA, following standard Southern blotting procedures. Preferably, the long probes of at least 100 nucleotides do not contain more than 1000 nucleotides. For long probes of at least 100 nucleotides in length, the carrier material is finally washed three times each for 15 minutes using 2×SSC, 0.1 % SDS at 42° C. (very low stringency), preferably washed three times each for 15 minutes using 0.5×SSC, 0.1% SDS at 42° C. (low stringency), more preferably washed three times each for 15 minutes using 0.2×SSC, 0.1% SDS at 42° C. (medium stringency), even more preferably washed three times each for 15 minutes using 0.2×SSC, 0.1% SDS at 55° C. (medium-high stringency), most preferably washed three times each for 15 minutes using 0.1×SSC, 0.1% SDS at 60° C. (high stringency), in particular washed three times each for 15 minutes using 0.1×SSC, 0.1% SDS at 68° C. (very high stringency).

Although not particularly preferred, it is contemplated that shorter probes, e.g. probes which are from about 15 to 99 nucleotides in length, such as from about 15 to about 70 nucleotides in length, may be also be used. For such short probes, stringency conditions are defined as prehybridization, hybridization, and washing post-hybridization at 5° C. to 10° C. below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1X Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to 99 nucleotides in length, the carrier material is washed once in 6×SCC plus 0.1% SDS for 15 minutes and twice each for 15 minutes using 6×SSC at 5° C. to 10° C. below the calculated Tm.

A DNA “coding sequence” or an “open reading frame (ORF)” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

An “expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway. A preferred secretory signal for the purposes of this invention is the signal sequence shown in SEQ ID NO: 2.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A chromosomal gene is rendered non-functional if the polypeptide that the gene encodes can no longer be expressed in a functional form. Such non-functionality of a gene can be induced by a wide variety of genetic manipulations as known in the art, some of which are described in Sambrook et al. vide supra. Partial deletions within the ORF of a gene will often render the gene non-functional, as will mutations.

“Operably linked”, when referring to DNA segments, indicates that the segments are arranged so that they function in concert for their intended purposes, e.g. transcription initiates in the promoter and proceeds through the coding segment to the terminator.

A coding sequence is “under the control” of transcriptional and translational control sequences in a cell when RNA polymerase transcribes the coding sequence into mRNA, which is then trans-RNA spliced and translated into the protein encoded by the coding sequence.

“Heterologous” DNA refers to DNA not naturally located in the cell, or in a chromosomal site of the cell. Preferably, the heterologous DNA includes a gene foreign to the cell.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a polypeptide of interest. The construct may optionally contain other nucleic acid segments.

The nucleic acid construct of the invention encoding the polypeptide of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleic acid construct of the invention encoding the polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques. The nucleic acid construct may also be prepared by polymerase chain reaction using specific primers, for instance as described in U.S. Pat. No. 4,683,202 or Saiki et al., Science 239 (1988), 487-491.

The term nucleic acid construct may be synonymous with the term “expression cassette” when the nucleic acid construct contains the control sequences necessary for expression of a coding sequence of the present invention

The term “control sequences” is defined herein to include all components which are necessary or advantageous for expression of the coding sequence of the nucleic acid sequence. Each control sequence may be native or foreign to the nucleic acid sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

The control sequence may be an appropriate promoter sequence, a nucleic acid sequence which is recognized by a host cell for expression of the nucleic acid sequence. The promoter sequence contains transcription and translation control sequences which mediate the expression of the polypeptide. The promoter may be any nucleic acid sequence which shows transcriptional activity in the host cell of choice and may be obtained from genes encoding extracellular or intracellular polypeptides either homologous or heterologous to the host cell.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region, which codes for an amino acid sequence linked to the amino terminus of the polypeptide which can direct the expressed polypeptide into the cell's secretory pathway of the host cell. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion of the exoprotein relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems.

Examples of suitable promoters for directing the transcription of the gene(s) of the present invention, especially in a bacterial host cell, are the promoters obtained from the E. coli lac operon, the Streptomyces coelicolor agarase gene (dagA), the Bacillus subtilis levansucrase gene (sacB), the Bacillus subtilis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75:3727-3731), as well as the tac promoter (DeBoer et al., 1983, Proceedings of the National Academy of Sciences USA 80:21-25). Further promoters are described in “Useful proteins from recombinant bacteria” in Scientific American, 1980, 242:74-94; and in Sambrook et al., 1989, supra.

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57:109-137.

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression, and possibly secretion.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Antibiotic selectable markers confer antibiotic resistance to such antibiotics as ampicillin, kanamycin, chloramphenicol, tetracycline, neomycin, hygromycin or methotrexate. Suitable markers for yeast host cells are ADE2, HIS3, LEU2, LYS2, MET3, TRP1, and URA3.

The vectors of the present invention preferably contain an element(s) that permits stable integration of the vector, or of a smaller part of the vector, into the host cell genome or autonomous replication of the vector in the cell independent of the genome of the cell.

The vectors, or smaller parts of the vectors such as amplification units of the present invention, may be integrated into the host cell genome when introduced into a host cell. For chromosomal integration, the vector may rely on the nucleic acid sequence encoding the polypeptide or any other element of the vector for stable integration of the vector into the genome by homologous or nonhomologous recombination.

Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences; specific examples of encoding sequences suitable for site-specific integration by homologous recombination are given in WO 02/00907 (Novozymes, Denmark), which is hereby incorporated by reference in its totality.

On the other hand, the vector may be integrated into the genome of the host cell by non-homologous recombination. These nucleic acid sequences may be any sequence that is homologous with a target sequence in the genome of the host cell, and, furthermore, may be non-encoding or encoding sequences. The copy number of a vector, an expression cassette, an amplification unit, a gene or indeed any defined nucleotide sequence is the number of identical copies that are present in a host cell at any time. A gene or another defined chromosomal nucleotide sequence may be present in one, two, or more copies on the chromosome. An autonomously replicating vector may be present in one, or several hundred copies per host cell.

An amplification unit of the invention is a nucleotide sequence that can integrate into the chromosome of a host cell, whereupon it can increase in number of chromosomally integrated copies by duplication of multiplication. The unit comprises an expression cassette as defined herein comprising at least one copy of a gene of interest and an expressable copy of a chromosomal gene, as defined herein, of the host cell. When the amplification unit is integrated into the chromosome of a host cell, it is defined as that particular region of the chromosome which is prone to being duplicated by homologous recombination between two directly repeated regions of DNA. The precise border of the amplification unit with respect to the flanking DNA is thus defined functionally, since the duplication process may indeed duplicate parts of the DNA which was introduced into the chromosome as well as parts of the endogenous chromosome itself, depending on the exact site of recombination within the repeated regions. This principle is illustrated in Janniére et al. (1985, Stable gene amplification in the chromosome of Bacillus subtilis. Gene, 40: 47-55), which is incorporated herein by reference.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMbeta1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

The present invention also relates to recombinant host cells, comprising a nucleic acid sequence of the invention, which are advantageously used in the recombinant production of the polypeptides. The term “host cell” encompasses any progeny of a parent cell which is not identical to the parent cell due to mutations that occur during replication.

The cell is preferably transformed with a vector comprising a nucleic acid sequence of the invention followed by integration of the vector into the host chromosome. “Transformation” means introducing a vector comprising a nucleic acid sequence of the present invention into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination as described above.

The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

The transformed or transfected host cells described above are cultured in a suitable nutrient medium under conditions permitting the expression of the desired polypeptide, after which the resulting polypeptide is recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

The polypeptide is recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989).

In the present context, the term “substantially pure polypeptide” means a polypeptide preparation which contains at the most 10% by weight of other polypeptide material with which it is natively associated (lower percentages of other polypeptide material are preferred, e.g. at the most 8% by weight, at the most 6% by weight, at the most 5% by weight, at the most 4% at the most 3% by weight, at the most 2% by weight, at the most 1 % by weight, and at the most ½% by weight). Thus, it is preferred that the substantially pure polypeptide is at least 92% pure, i.e. that the polypeptide constitutes at least 92% by weight of the total polypeptide material present in the preparation, and higher percentages are preferred such as at least 94% pure, at least 95% pure, at least 96% pure, at least 96% pure, at least 97% pure, at least 98% pure, at least 99%, and at the most 99.5% pure. The polypeptides disclosed herein are preferably in a substantially pure form. In particular, it is preferred that the polypeptides disclosed herein are in “essentially pure form”, i.e. that the polypeptide preparation is essentially free of other polypeptide material with which it is natively associated. This can be accomplished, for example, by preparing the polypeptide by means of well-known recombinant methods. Herein, the term “substantially pure polypeptide” is synonymous with the terms “isolated polypeptide” and “polypeptide in isolated form”.

In the present context, the homology between two amino acid sequences or between two nucleotide sequences is described by the parameter “identity”. For purposes of the present invention, alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98).

Multiple alignments of protein sequences may be made using “ClustalW” (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680). Multiple alignment of DNA sequences may be done using the protein alignment as a template, replacing the amino acids with the corresponding codon from the DNA sequence.

In the present context, the term “allelic variant” denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene. Allelic variants are included in the present definition of functional homologues.

The S2A/S1E protease or functional homologue thereof may be a wild-type protein identified and isolated from a natural source. Such wild-type proteases may be specifically screened for by standard techniques known in the art. Furthermore, genes encoding the S2A/S1E protease, or a functional homologue thereof, may be prepared by a DNA shuffling technique, such as described in J. E. Ness et al. Nature Biotechnology 17, 893-896 (1999). Moreover, the S2A/S1E protease, or functional homologue thereof, may be an artificial variant. Such artificial variants may be constructed by standard techniques known in the art, such as by site-directed/random mutagenesis. In one embodiment of the invention, amino acid changes (in the artificial variant as well as in wild-type polypeptides) are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine, valine and methionine), aromatic amino acids (phenylalanine, tryptophan and tyrosine), and small amino acids (glycine, alanine, serine and threonine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

It will be apparent to those skilled in the art that such modifications can be made outside the regions critical to the function of the molecule and still result in an active polypeptide. Amino acid residues essential to the activity of the polypeptide encoded by the nucleotide sequence of the invention, and therefore preferably not subject to modification, such as substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

Moreover, a nucleotide sequence encoding a polypeptide of the present invention may be modified by introduction of nucleotide substitutions which do not give rise to another amino acid sequence of the polypeptide encoded by the nucleotide sequence, but which correspond to the codon usage of the host organism intended for production of the enzyme.

The introduction of a mutation into the nucleotide sequence to exchange one nucleotide for another nucleotide may be accomplished by site-directed mutagenesis using any of the methods known in the art. Particularly useful is the procedure, which utilizes a supercoiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemimethylated DNA to digest the parental DNA template and to select for mutation-containing synthesized DNA. Other procedures known in the art may also be used. For a general description of nucleotide substitution, see, e.g., Ford et al., 1991, Protein Expression and Purification 2: 95-107.

### DETAILED DESCRIPTION

In particular embodiments, the proteases of the invention and for use according to the invention are selected from the group consisting of:

(a) proteases belonging to the EC 3.4.-.- enzyme group;

(b) Serine proteases belonging to the S group of the above Handbook;

(c1) Serine proteases of peptidase family S2A;

(c2) Serine proteases of peptidase family S1E as described in Biochem. J. 290:205-218 (1993) and in MEROPS a protease database, release 6.20, Mar. 24, 2003, (www.merops.ac.uk). The database is described in Rawlings, N.D., O'Brien, E. A. & Barrett, A. J. (2002) MEROPS: the protease database. Nucleic Acids Res. 30, 343-346.

For determining whether a given protease is a Serine protease, and a family S2A protease, reference is made to the above Handbook and the principles indicated therein. Such determination can be carried out for all types of proteases, be it naturally occurring or wild-type proteases; or genetically engineered or synthetic proteases.

Protease activity can be measured using any assay, in which a substrate is employed, that includes peptide bonds relevant for the specificity of the protease in question. Assay-pH and assay-temperature are likewise to be adapted to the protease in question. Examples of assay-pH-values are pH 2, 3, 4, 5, 6, 7, 8, 9, 10, 11, or 12. Examples of assay-temperatures are 30, 35, 37, 40, 45, 50, 55, 60, 65, 70, 80, 90, or 95° C.

Examples of protease substrates are casein, such as Azurine-Crosslinked Casein (AZCL-casein). For the purposes of this invention, S2A protease activity is preferably measured using the PNA assay with succinyl-alanine-alanine-proline-phenylalnine-paranitroanilide as a substrate unless otherwise mention. The principle of the PNA assay is described in Rothgeb, T. M., Goodlander, B. D., Garrison, P. H., and Smith, L. A., Journal of the American Oil Chemists' Society, Vol. 65 (5) pp. 806-810 (1988).

There are no limitations on the origin of the protease of the invention and/or for use according to the invention. Thus, the term protease includes not only natural or wild-type proteases obtained from microorganisms of any genus, but also any mutants, variants, fragments etc. thereof exhibiting protease activity, as well as synthetic proteases, such as shuffled proteases, and consensus proteases. Such genetically engineered proteases can be prepared as is generally known in the art, eg by Site-directed Mutagenesis, by PCR (using a PCR fragment containing the desired mutation as one of the primers in the PCR reactions), or by Random Mutagenesis. The preparation of consensus proteins is described in eg EP 897985.

In a specific embodiment, the protease is a low-allergenic variant, designed to invoke a reduced immunological response when exposed to animals, including man. The term immunological response is to be understood as any reaction by the immune system of an animal exposed to the protease. One type of immunological response is an allergic response leading to increased levels of IgE in the exposed animal. Low-allergenic variants may be prepared using techniques known in the art. For example the protease may be conjugated with polymer moieties shielding portions or epitopes of the protease involved in an immunological response. Conjugation with polymers may involve in vitro chemical coupling of polymer to the protease, e.g. as described in WO 96/17929, WO 98/30682, WO 98/35026, and/or WO 99/00489. Conjugation may in addition or alternatively thereto involve in vivo coupling of polymers to the protease. Such conjugation may be achieved by genetic engineering of the nucleotide sequence encoding the protease, inserting consensus sequences encoding additional glycosylation sites in the protease and expressing the protease in a host capable of glycosylating the protease, see e.g. WO 00/26354. Another way of providing low-allergenic variants is genetic engineering of the nucleotide sequence encoding the protease so as to cause the protease to self-oligomerize, effecting that protease monomers may shield the epitopes of other protease monomers and thereby lowering the antigenicity of the oligomers. Such products and their preparation is described e.g. in WO 96/16177. Epitopes involved in an immunological response may be identified by various methods such as the phage display method described in WO 00/26230 and WO 01/83559, or the random approach described in EP 561907. Once an epitope has been identified, its amino acid sequence may be altered to produce altered immunological properties of the protease by known gene manipulation techniques such as site directed mutagenesis (see e.g. WO 00/26230, WO 00/26354 and/or WO 00/22103) and/or conjugation of a polymer may be done in sufficient proximity to the epitope for the polymer to shield the epitope.

The first aspect of the invention is detailed in the summary above, but, among other things, it relates to methods of producing heterologous S2A/S1E proteases by using Gram-positive host cells comprising at least one polynucleotide encoding at least one S2A or S1E protease, wherein the codon usage in the coding part of at least one polynucleotide corresponds to the average codon usage in a Bacillus cell, and wherein the G/C content is adjusted by replacing G/C-rich codons with alternatives, while remaining close to the average codon-usage of the cell.

The sequence information from B. licheniformis ATCC 14580 published in WO 02/29113, which is incorporated herein by reference, may be used to generate suitable codon usage tables as outlined herein for expression in Bacillus licheniformis.

For improved expression in Bacillus subtilis of heterologous sequences, it may be an advantage to approximate the codon usage based on the Bacillus subtilis chromosomal sequence, which is publicly available (Kunst, F, et al., The Complete Genome Sequence of the Gram-positive . . . , 1997, Nature, 390, pp: 249-256).

The codon usage tables can be based on (1) the codons used in all the open reading frames, (2) selected open reading frames, (3) fragments of the open reading frames, or (4) fragments of selected open reading frames, preferably the fragments encode the N-terminal amino acids of the encoded polypeptide, and more preferably at least the 20 first N-terminal amino acids.

Synthetic genes can be designed with only the most preferred codon for each amino acid; with a number of common codons for each amino acid; or with the same or similar statistical average frequencies of codon usages found in the table of choice.

The synthetic gene can be constructed using any method such as site-directed mutagenesis or PCR generated mutagenesis in accordance with methods known in the art. Although, in principle, the modification may be performed in vivo, i.e., directly on the cell expressing the nucleotide sequence to be modified, it is preferred that the modification is performed in vitro.

The synthetic gene can be further modified by operably linking the synthetic gene to one or more control sequences which direct the expression of the coding sequence in a suitable host cell under conditions compatible with the control sequences using the methods described herein. Nucleic acid constructs, recombinant expression vectors, and recombinant host cells comprising the synthetic gene can also be prepared using the methods described herein.

All the expressed genes in the following examples are integrated by homologous recombination on the Bacillus host cell genome. The genes are expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The gene coding for Chloramphenicol acetyl-transferase was used as marker. (Described in eg. Diderichsen, B.; Poulsen, G. B.; Joergensen, S. T.; A useful cloning vector for Bacillus subtilis. Plasmid 30:312 (1993)).

The first aspect of the invention relates to a method of producing a heterologous S2A/S1E protease in a Gram-positive host cell, the method comprising the steps of:
(a) cultivating in a fed-batch fermentation a Gram-positive cell comprising at least one polynucleotide encoding the heterologous S2A/S1E protease under conditions conducive for production of the protease, wherein at least 20%, more preferably at least 50%, of the duration of said cultivating step takes place at a temperature of below 36.5° C.; preferably at a temperature of below 36° C.; more preferably at a temperature of below 35° C., even more preferably below 33° C., or most preferably at a temperature of below 31° C.; and
(b) recovering the protease.

The inventors found that it was of some advantage if the cultivating step in the method of the invention was “kick-started” at the usual 37° C. for a bried period, until the Gram-positive host cells were actively growing, whereup they lowered the temperature for the remainder of the fermentation to achieve improved S2A/S1E protease yields. Non-limiting examples of this temperature-shift strategy are provided in the examples section below.

Accordingly, a preferred embodiment relates to a method of the invention, wherein the first 50% or less of the duration of said cultivating step takes place at a temperature of above 31° C.; preferably the first 40% or less of the duration of said cultivating step; more preferably the first 30% or less; or most preferably the first 20% or less of the duration of the cultivating step takes place at a temperature of above 31° C.; preferably at a temperature of above 33° C.; more preferably above 35° C.; or most preferably above 36° C.

In a preferred embodiment the Gram-positive cell is a Bacillus cell, preferably a Bacillus species chosen from the group consisting of Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis.

Four specific synthetic polynucleotides of the first aspect encoding S2A/S1E proteases are provided herewith in SEQ ID NO's: 3, 35, 37, and 39.

Accordingly, a preferred embodiment relates to the polynucleotide of the invention which comprises a nucleotide sequence at least 70%, 75%, 80%, preferably 85%, more preferably 90%, still more preferably 95%, more preferably 97%, more preferably 98%, still more preferably 99%, and most preferably 99.5% identical to the nucleotide sequence shown in positions 577 to 1140 of SEQ ID NO's: 3, 35, 37, or 39.

Another preferred embodiment relates to the polynucleotide of the invention which comprises a nucleotide sequence at least 70%, 75%, 80%, preferably 85%, more preferably 90%, still more preferably 95%, more preferably 97%, more preferably 98%, still more preferably 99%, and most preferably 99.5% identical to the nucleotide sequence shown in positions 577 to 1140 of SEQ ID NO: 3; in positions 526 to 1089 of SEQ ID NO: 5; in positions 508 to 1083 of SEQ ID NO: 9; in positions 519 to 1085 of SEQ ID NO: 13; in positions 568 to 1143 of SEQ ID NO: 17; in positions 574 to 1149 of SEQ ID NO: 19; in positions 574 to 1149 of SEQ ID NO: 21; in positions 586 to 1152 of SEQ ID NO: 23; in positions 586 to 1149 of SEQ ID NO: 25; in positions 586 to 1152 of SEQ ID NO: 27; in positions 502 to 1065 of SEQ ID NO: 29; in positions 496 to 1059 of SEQ ID NO: 31; in positions 499 to 1062 of SEQ ID NO: 33; in positions 577 to 1140 of SEQ ID NO: 35; in positions 577 to 1140 of SEQ ID NO: 37; or in positions 577 to 1140 of SEQ ID NO: 39.

Preferred S2A/S1E proteases of the invention are provided in SEQ ID NO's: 4, 6, 10, 14, 18, 20, 22, 24, 26, 28, 30, 32, and 34. Therefore, a preferred S2A/S1E protease comprises an amino acid sequence at least 70%, 75%, 80%, preferably 85%, more preferably 90%, still more preferably 95%, more preferably 97%, more preferably 98%, still more preferably 99%, and most preferably 99.5% identical to the amino acid sequence of the mature part of the polypeptide shown in SEQ ID NO's: 4, 6, 10, 14, 18, 20, 22, 24, 26, 28, 30, 32, or 34.

Other preferred S2A or S1E proteases of the invention are derived from one or more Nocardiopsis species chosen from the group consisting of Nocardiopsis sp. NRRL 18262, Nocardiopsis dassonvillei subsp. dassonvillei DSM 43235, Nocardiopsis Alba DSM 15647, Nocardiopsis prasina DSM 15648, Nocardiopsis prasina DSM 15649, Nocardiopsis prasina (previously alba) DSM 14010, Nocardiopsis sp. DSM 16424, Nocardiopsis alkaliphila DSM 44657, and Nocardiopsis lucentensis DSM 44048.

As mentioned above, genome sequences of Bacillus licheniformis and Bacillus subtilis were available to the present inventors, and they were both used for the construction of codon-usage data.

In a preferred embodiment, the codon usage in at the least one encoding polynucleotide of the invention corresponds to the average codon usage in a Bacillus cell, preferably a Bacillus licheniformis or a Bacillus subtilis cell, and more preferably a Bacillus licheniformis ATCC 14580 cell.

A preferred embodiment relates to a polynucleotide of the invention, wherein the codon usage corresponds to the average codon usage in one or more polynucleotide encoding one or more secreted polypeptide endogenous to the Gram-positive Bacillus cell; preferably to the average codon usage in at least the first 5, preferably 10, more preferably 15, even more preferably 20, and most preferably at least the first 25 codon triplets of one or more polynucleotide encoding one or more secreted polypeptide endogenous to the Bacillus cell; preferably the codon triplets of ten or more polynucleotides encoding ten or more secreted polypeptides endogenous to the Bacillus cell.

Deposit of Biological Material

The following biological materials have been deposited under the terms of the Budapest Treaty with the DSMZ (Deutsche Sammlung von Mikroorganismen und Zellkulturen GmbH, MascheroderWeg 1b, D-38124 Braunschweig, Germany), and given the following accession numbers:

| Deposit | Accession Number | Date of Deposit |
|---|---|---|
| Nocardiopsis sp. | DSM 16424 | May 24, 2004 |
| Nocardiopsis prasina | DSM 15649 | May 30, 2003 |
| Nocardiopsis prasina | DSM 14010 | Jan. 20, 2001 |
| (previously alba) | | |

These strains have been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

Strain DSM 15649 was isolated in 2001 from a soil sample from Denmark.

The following strains are publicly available from DSMZ:

| | |
|---|---|
| Nocardiopsis dassonvillei subsp. dassonvillei | DSM 43235 |
| Nocardiopsis alkaliphila | DSM 44657 |
| Nocardiopsis lucentensis | DSM 44048 |

Nocardiopsis dassonvillei subsp. dassonvillei strain DSM 43235 was also deposited at other depositary institutions as follows: ATCC 23219, IMRU 1250, NCTC 10489.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention described and claimed herein is not to be limited in scope by the specific embodiments herein disclosed, including the following examples, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

### Example 1

Construction of Strains

Strains used: Bacillus subtilis MB1053 (W0200395658)
Media used: TY: (As described in Ausubel, F. M. et al. (eds.) “Current protocols in Molecular Biology”. John Wiley and Sons, 1995).

All the expressed genes in the following examples are integrated by homologous recombination on the Bacillus subtilis MB1053 host cell genome (WO200395658). The genes are expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The gene coding for chloramphenicol acetyl-transferase was used as marker. (Described in eg. Diderichsen,B.; Poulsen,G. B.; Joergensen,S. T.; A useful cloning vector for Bacillus subtilis. Plasmid 30:312 (1993)).

Construction of Bacillus subtilis Strains Sav-1 ORS, Sav-L2, Sav-L1 and Sav-L3

A synthetic 10R gene (1ORS) encoding a S2A (or S1E) protease denoted 10R from Nocardiopsis sp. NRRL 18262 (WO 01/58276) was constructed. This synthetic gene was fused by PCR in frame to the DNA (shown in SEQ ID NO:1) coding for the signal peptide (shown in SEQ ID NO:2) from SAVINASE™ a well-known commercial protease derived from Bacillus clausii (Novozymes, Denmark) resulting in the coding sequence Sav-10RS, which is shown in SEQ ID NO: 3. The fusion sequence was integrated into a Bacillus subtilis host cell and the resulting strain was denoted Sav-10RS.

An analogous Bacillus subtilis strain was made with the DNA coding for the pro-form of a S1E protease from Nocardiopsis dassonvillei subsp. Dassonvillei DSM 43235, denoted L2, fused by PCR in frame to the DNA coding for the signal peptide from SAVINASE™ (Novozymes) the resulting strain was denoted Bacillus subtilis Sav-L2. The DNA sequence including the partial Savinase signal fused with the coding region for the pro-mature L2 protease is shown in SEQ ID NO: 5, as amplified with primers 1423 (SEQ ID NO: 7) and 1475 (SEQ ID NO: 8).

1423 (SEQ ID NO: 7):
gcttttagttcatcgatcgcatcggctgctccggcccccgtcccccag
1475 (SEQ ID NO: 8):
ggagcggattgaacatgcgattaggtccggatcctgacaccccag

A Bacillus subtilis strain was also made with the DNA coding for the pro-form of a S1E protease from Nocardiopsis dassonvillei subsp. Dassonvillei DSM 43235, denoted L1, fused by PCR in frame to the DNA coding for the signal peptide from SAVINASE™ (Novozymes, Denmark), the resulting strain was denoted Bacillus subtilis Sav-L1. The DNA sequence including the partial Savinase signal fused with the coding region for the pro-mature L1 protease is shown in SEQ ID NO: 9, as amplified with primers 1485 (SEQ ID NO: 11) and 1424 (SEQ ID NO: 12).

1485 (SEQ ID NO: 11):
ggagcggatgaacatgcgattactaaccggtcaccagggacagc
1424 (SEQ ID NO: 12):
ggagcggatgaacatgcgattactaaccggtcaccagggacagc

A Bacillus subtilis strain was made with the DNA coding for the pro-form of a S1E protease from Nocardiopsis sp. DSM 16424, denoted L3, fused by PCR in frame to the DNA coding for the signal peptide from SAVINASE™ (Novozymes, Denmark), the resulting strain was denoted Bacillus subtilis Sav-L3. The DNA sequence including the partial Savinase signal fused with the coding region for the pro-mature L3 protease is shown in SEQ ID NO: 13, as amplified with primers 1718 (SEQ ID NO: 15) and 1720 (SEQ ID NO: 16).

1718 (SEQ ID NO: 15):
agttcatcgatcgcatcggctgcgcccggccccgtcccccag
1720 (SEQ ID NO: 16):
ggagcggattgaacatgcgatcagctggtgcggatgcgaac

The Sav-10RS, Sav-L1, Sav-L2 and Sav-L3 genes were integrated by homologous recombination on the Bacillus subtilis MB1053 host cell genome. Chloramphenicol resistant transformants were checked for protease activity on 1% skim milk LB-PG agar plates (supplemented with 6 μg/ml chloramphenicol). Some protease positive colonies were further analyzed by DNA sequencing of the insert to confirm the correct DNA sequence, and one strain for each construct was selected.

The four selected B. subtilis strains Sav-10RS, Sav-L2, Sav-L1, and Sav-L3 were fermented on a rotary shaking table in 500 ml baffled Erlenmeyer flasks containing 100 ml TY supplemented with 6 mg/l chloramphinicol. Four Erlenmeyer flasks for each of the four B. subtilis strains were fermented in parallel. Two of the four Erlenmeyer flasks were incubated at 37° C. (250 rpm) and two at 30° C. (250 rpm). A sample was taken from each shake flask on days 1, 2 and 3 and analyzed for proteolytic activity. For each strain the average for each set of two samples is presented in the tables below, relative to the average of the day one sample at 37° C.

TABLE 1
Proteolytic activity for Sav-10RS relative to day 1 at 37° C.

| | Day 1 | Day 2 | Day 3 |
|---|---|---|---|
| Sav-10RS 37° C. | 1.0 | 0.9 | 0.9 |
| Sav-10RS 30° C. | 6.8 | 5.9 | 5.4 |

TABLE 2
Proteolytic activity for Sav-L1 relative to day 1 at 37° C.

| | Day 1 | Day 2 | Day 3 |
|---|---|---|---|
| Sav-L1 37° C. | 1.0 | 1.3 | 0.9 |
| Sav-L1 30° C. | 1.4 | 1.7 | 1.8 |

TABLE 3
Proteolytic activity for Sav-L2 relative to day 1 at 37° C.

| | Day 1 | Day 2 | Day 3 |
|---|---|---|---|
| Sav-10L2 37° C. | 1.0 | 1.0 | 0.8 |
| Sav-10L2 30° C. | 1.4 | 1.7 | 1.5 |

TABLE 4
Proteolytic activity for Sav-L3 relative to day 1 at 37° C.

| | Day 1 | Day 2 | Day 3 |
|---|---|---|---|
| Sav-L3 37° C. | 1.0 | 0.7 | 0.6 |
| Sav-L3 30° C. | 1.5 | 1.3 | 1.2 |

As it can be seen from tables 1-4, the lower fermentation temperature of 30° C. increases the expression level of all four tested S2A/S1E Nocardiopsis sp. proteases when compared with 37° C.

Non-limiting examples of genes encoding S2A/S1E proteases suitable for expression and production by the methods of the invention are provided in SEQ ID NO's: 17, 19, 21, 23, 25, 27, 29, 31, and 33; the amino acid sequences of the encoded proteases are provided correspondingly in SEQ ID NO's: 18, 20, 22, 24, 26, 28, 30, 32, and 34.

### Example 2

Expression of a Synthetic 10 Protease Gene Using a Temperature Downshift

One strategy for designing a synthetic DNA sequence encoding a given amino acid sequence is denoted randomization. The starting point is the protein sequence, or a wildtype DNA sequence encoding the protein sequence, and a codon table. The codon table is prepared from coding DNA sequences selected from the genome of the production host or a related species, using all or a subset of the sequences. In this example, the codon table was then modified by removing the most rarely used codons and some rarely used codons with a high GC-content.

In this context a codon table is taken to mean a list of all possible 64 codons together with frequencies giving the relative use of a given codon relative the other codons encoding the same amino acid in the chosen subset of DNA sequences.

The codon table and the protein sequence were then used to generate a synthetic DNA sequence as follows. For any given amino acid a codon was chosen with a probability given by the frequency given in the codon table. A review of codon optimization methods is given in Claes Gustafsson, Sridhar Govindarajan and Jeremy Minshull: Codon bias and heterologous protein expression, article in press (available from www.sciencedirect.com), Trends in Biotechnology.

Another strategy for the design of a synthetic DNA sequence encoding a given protein sequence is called strict optimization. The starting point in strict optimization is also a protein sequence, or DNA sequence encoding the protein sequence, and a codon table. Doing strict optimization, only the codon with the highest frequency in the codon table is used to encode a given amino acid.

The randomization method will easily generate a large number of synthetic DNA sequences all encoding the same protein and all with approximately the same codon statistics as listed in the codon table used. A number of criteria can be used to select the final candidate for the gene.

We generated a number of synthetic modified genes (shown in SEQ ID NO's: 35, 37, and 39) encoding a S2A (or S1E) proteases from a Nocardiopsis sp. NRRL1 8262. For each gene the free energy of folding and minimum energy conformation was computed using the program RNAfold from the Vienna package described in Nucleic Acids Res. 31: 3429-3431 (2003). A gene was selected (SEQ ID NO: 35) and incorporated into the genome of a Bacillus host cell as a single copy in an exact identical construction as in a comparable strain expressing the same 10 R protease but from the wild type gene. The integrity of each chromosomal integrant was verified by DNA sequencing of the entire expression cassettes.

The two integrants were fermented in a number of shake flasks using rich media for up to 6 days under vigorously shaking at 37° C. for 24 hours followed by incubation at 26° C. (37/26). After incubations at the indicated temperatures for the indicated time, 1 ml supernatant samples were harvested by centrifugation and samples were analysed for protease. The results are presented in table 5 for the strains harbouring the synthetic protease gene relative to the strains harbouring the wildtype protease gene fermented under the exact same conditions.

At 37/26° C. expression of the 10R synthetic gene resulted in an increased level of protease activity with a factor of between 1.5 and 13. A very large variation in the expression is observed which is partly due to the lack of control over the pH during the fermentation. There is however no doubt that the synthetic gene lead to increased protease expression, in average approx. 5 times.

TABLE 5
Expression yields from using a synthetic protease
gene relative to a wt gene.

| Ferm. time | Strain | Rel. activity |
|---|---|---|
| 4 days | Protease 2-5a | 1.6 |
| 4 days | Protease 2-8a | 3.7 |
| 5 days | Protease 2-5a | 1.5 |
| 5 days | Protease 2-8a | 3.6 |
| 6 days | Protease 2-5a | 2.3 |
| 6 days | Protease 2-8a | 5.6 |
| 5 days | Protease 2-8a | 5.8 |
| 6 days | Protease 2-8a | 4.1 |
| 5 days | Protease 2-8a | 5.8 |
| 6 days | Protease 2-8a | 9.6 |
| 5 days | Protease A1-8 | 10.6 |
| 6 days | Protease A1-8 | 13.3 |

1-10. (canceled)

11. A method of producing a heterologous S2A/S1E protease in a Gram-positive host cell, the method comprising the steps of:
(a) cultivating in a fed-batch fermentation a Gram-positive cell comprising at least one polynucleotide encoding the heterologous S2A/S1E protease under conditions conducive for production of the protease, wherein the first 50% or less of the duration of said cultivating step takes place at a temperature above 31° C. whereafter the temperature is lowered and at least 20% of the duration of said cultivating takes place at a temperature below 36.5° C.; and
(b) recovering the protease.

12. The method of claim 11, wherein the Gram-positive host cell is a Bacillus cell.

13. The method of claim 11, wherein the Gram-positive host cell is a Bacillus species chosen from the group consisting of Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis.

14. The method of claim 11, wherein the S2A/S1E protease comprises an amino acid sequence which is the mature part of the polypeptide shown in SEQ ID NO: 4, SEQ ID NO: 6, SEQ ID NO: 10, SEQ ID NO: 14, SEQ ID NO: 18; SEQ ID NO: 20; SEQ ID NO: 22; SEQ ID NO: 24; SEQ ID NO: 26; SEQ ID NO: 28; SEQ ID NO: 30; SEQ ID NO: 32; or SEQ ID NO: 34.

15. The method of claim 11, wherein the S2A/S1E protease is derived from a Nocardiopsis species chosen from the group consisting of Nocardiopsis sp. NRRL 18262, Nocardiopsis dassonvillei subsp. dassonvillei DSM 43235, Nocardiopsis Alba DSM 15647, Nocardiopsis prasina DSM 15618, Nocardiopsis prasina DSM 15649, Nocardiopsis prasina (previously alba) DSM 14010, Nocardiopsis sp. DSM 16424, Nocardiopsis alkaliphila DSM 44657, and Nocardiopsis lucentensis DSM 44048.

16. The method of claim 11, wherein the at least one polynucleotide comprises a nucleotide sequence selected from the group consisting of the nucleotide sequence shown in positions 577 to 1140 of SEQ ID NO: 3; in positions 526 to 1089 of SEQ ID NO: 5; in positions 508 to 1083 of SEQ ID NO: 9; in positions 519 to 1085 of SEQ ID NO: 13; in positions 568 to 1143 of SEQ ID NO: 17; in positions 574 to 1149 of SEQ ID NO: 19; in positions 574 to 1149 of SEQ ID NO: 21; in positions 586 to 1152 of SEQ ID NO: 23; in positions 586 to 1149 of SEQ ID NO: 25; in positions 586 to 1152 of SEQ ID NO: 27; in positions 502 to 1065 of SEQ ID NO: 29; in positions 496 to 1059 of SEQ ID NO: 31; in positions 499 to 1062 of SEQ ID NO: 33; in positions 577 to 1140 of SEQ ID NO: 35; in positions 577 to 1140 of SEQ ID NO: 37; or in positions 577 to 1140 of SEQ ID NO: 39.

17. The method of claim 11, wherein the codon usage in the at least one polynucleotide corresponds to the average codon usage in a Bacillus cell.

18. The method of claim 11, wherein at least 50% of the duration of said cultivating step takes place at a temperature below 36.5° C.

19. The method of claim 11, wherein the first 40% or less of the duration of said cultivating step takes place at a temperature above 31° C.

20. The method of claim 19, wherein the first 50% or less of the duration of the cultivating step takes place at a temperature above 33° C.


SEQUENCE LISTING (40 sequences)

SEQ ID NO: 1 - DNA, 81 bp, Bacillus clausii
  CDS (1)..(81)
  sig_peptide (1)..(81)
atgaagaaaccgttggggaaaattgtcgcaagcaccgcactactcatttctgttgcttttagttcatcgatcgcatcggct

SEQ ID NO: 2 - protein, 27 aa, Bacillus clausii
MKKPLGKIVASTALLISVAFSSSIASA

SEQ ID NO: 3 - DNA, 1143 bp, Artificial sequence
  Fusion of Savinase signal with synthetic S2A protease
  CDS (1)..(1140)
  sig_peptide (1)..(81): Savinase signal
  mat_peptide (577)..(1140)
atgaagaaaccgttggggaaaattgtcgcaagcaccgcactactcatttctgttgcttttagttcatcgatcgcatcggctgctactggagcattacctcagtctcctacacctgaagcagatgcagtatcgatgcaagaagcattacaacgtgatcttgatcttacatcagctgaagctgaggaattacttgctgcacaagatacagcctttgaagttgatgaagctgccgctgaagcagctggtgatgcatatggtggttcagtattcgatactgaatcactcgaacttactgtactagtgaccgatgcagcagctgttgaagctgttgaagccacaggtgcaggtacagagctcgtatcttatggtattgatggattagatgagatcgtacaagagcttaatgcagctgatgccgttccaggtgtagttggatggtatcctgatgtagcaggtgatactgttgtcttagaagttcttgaaggctctggagctgatgtttctggacttttagcagacgcaggagtcgatgcatccgcggttgaagtgaccacgtcagatcagcctgaactctatgccgatatcattggaggcctagcgtacacaatgggtggtcgctgcagcgtaggatttgcagccacaaatgcagctggacaacctggcttcgtgacagctggacattgcggccgcgtcggtacacaggttactatcggcaatggaagaggtgtctttgagcaaagcgtatttcccgggaatgatgctgccttcgttagaggtacgtccaactttacgcttactaacttagtatctagatacaacactggcggatatgcaactgtagcaggtcacaatcaagcacctattggctctagcgtctgccgctcagggtcgactacaggatggcattgtggaaccattcaagctagaggtcagagcgtgagctatcctgaaggtaccgtaacgaacatgactcgtacgactgtatgtgcagaaccaggtgactctggaggttcatatatcagcggtacgcaagcgcaaggcgttacctcaggtggatccggtaactgtaggacaggtggcacaacgttctaccaggaagtgacaccgatggtgaactcttggggagttagactccgtacataa

SEQ ID NO: 4 - protein, 380 aa, Artificial sequence
  Synthetic Construct
MKKPLGKIVASTALLISVAFSSSIASAATGALPQSPTPEADAVSMQEALQRDLDLTSAEAEELLAAQDTAFEVDEAAAEAAGDAYGGSVFDTESLELTVLVTDAAAVEAVEATGAGTELVSYGIDGLDEIVQELNAADAVPGVVGWYPDVAGDTVVLEVLEGSGADVSGLLADAGVDASAVEVTTSDQPELYADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 5 - DNA, 1112 bp, Artificial sequence
  Fusion of Savinase signal with S2A protease gene
  CDS (1)..(1089)
  sig_peptide (1)..(27): Partial Savinase signal peptide
  mat_peptide (526)..(1089)
gcttttagttcatcgatcgcatcggctgctccggcccccgtcccccagacccccgtcgccgacgacagcgccgccagcatgaccgaggcgctcaagcgcgacctcgacctcacctcggccgaggccgaggagcttctctcggcgcaggaagccgccatcgagaccgacgccgaggccaccgaggccgcgggcgaggcctacggcggctcactgttcgacaccgagaccctcgaactcaccgtgctggtcaccgacgcctccgccgtcgaggcggtcgaggccaccggagcccaggccaccgtcgtctcccacggcaccgagggcctgaccgaggtcgtggaggacctcaacggcgccgaggttcccgagagcgtcctcggctggtacccggacgtggagagcgacaccgtcgtggtcgaggtgctggagggctccgacgccgacgtcgccgccctgctcgccgacgccggtgtggactcctcctcggtccgggtggaggaggccgaggaggccccgcaggtctacgccgacatcatcggcggcctggcctactacatgggcggccgctgctccgtcggcttcgccgcgaccaacagcgccggtcagcccggtttcgtcaccgccggccactgcggcaccgtcggcaccggcgtgaccatcggcaacggcaccggcaccttccagaactcggtcttccccggcaacgacgccgccttcgtccgcggcacctccaacttcaccctgaccaacctggtctcgcgctacaactccggcggctaccagtcggtgaccggtaccagccaggccccggccggctcggccgtgtgccgctccggctccaccaccggctggcactgcggcaccatccaggcccgcaaccagaccgtgcgctacccgcagggcaccgtctactcgctcacccgcaccaacgtgtgcgccgagcccggcgactccggcggttcgttcatctccggctcgcaggcccagggcgtcacctccggcggctccggcaactgctccgtcggcggcacgacctactaccaggaggtcaccccgatgatcaactcctggggtgtcaggatccggacctaatcgcatgttcaatccgctcc

SEQ ID NO: 6 - protein, 363 aa, Artificial sequence
  Synthetic Construct
AFSSSIASAAPAPVPQTPVADDSAASMTEALKRDLDLTSAEAEELLSAQEAAIETDAEATEAAGEAYGGSLFDTETLELTVLVTDASAVEAVEATGAQATVVSHGTEGLTEVVEDLNGAEVPESVLGWYPDVESDTVVVEVLEGSDADVAALLADAGVDSSSVRVEEAEEAPQVYADIIGGLAYYMGGRCSVGFAATNSAGQPGFVTAGHCGTVGTGVTIGNGTGTFQNSVFPGNDAAFVRGTSNFTLTNLVSRYNSGGYQSVTGTSQAPAGSAVCRSGSTTGWHCGTIQARNQTVRYPQGTVYSLTRTNVCAEPGDSGGSFISGSQAQGVTSGGSGNCSVGGTTYYQEVTPMINSWGVRIRT

SEQ ID NO: 7 - DNA, 48 bp, Artificial sequence
  Primer 1423
gcttttagttcatcgatcgcatcggctgctccggcccccgtcccccag

SEQ ID NO: 8 - DNA, 45 bp, Artificial sequence
  Primer 1475
ggagcggattgaacatgcgattaggtccggatcctgacaccccag

SEQ ID NO: 9 - DNA, 1108 bp, Artificial sequence
  Fusion of Savinase signal with S2A protease gene
  CDS (1)..(1083)
  sig_peptide (1)..(27): Partial Savinase signal
  mat_peptide (508)..(1083)
gcttttagttcatcgatcgcatcggctgcgaccgtaccggccgagccagcgagcgaggcccagacgatgatggaagcgctgcagagagacctcggcctcaccccgctcggggccgaggagctgctctcggcgcaggaagaggcgatcgagaccgacgccgaggccaccgaggccgcgggagcgtcctacggcggctccctgttcgacaccgagaccctccagctcaccgtgctggtgaccgacgcctcggccgtcgaggcggtggaggccaccggcgccgaggccaccgtggtctcacacggcgcagagggcctggccgaggtggtcgacgcgctcgacgagaccggcggccgggaaggggtcgtcggctggtacccggacgtggagagcgacaccgtcgtggtccaggtcgccgagggcgccagcgccgacggcctcatcgaggccgcgggcgtggacccctccgccgtccgggtggaggagaccagtgagactccgcgcctgtacgccgacatcgtcggcggcgaggcgtactacatgggcggcggacgctgctcggtcgggttcgccgtgaccgacggctccggcgcgggcggcttcgtgacggcgggccactgcggcaccgtcggcaccggcgccgagagctccgacggcagcggctccggaaccttccaggagtccgtcttcccgggcagcgacggcgccttcgtcgcggccacctccaactggaacgtgaccaacctggtcagccggtacgactccggcagcccccaggcggtgtcgggttccagccaggccccggagggctcggcggtgtgccgctccggctccaccaccggctggcactgcgggaccatcgaggcccgcggccagacggtgaactacccgcagggcacggtccaggacctgacccggacggacgtgtgcgccgagcccggtgactccggcggctcgttcatcgccggttcgcaggcccagggcgtcacctccggcggctcgggcaactgcacctccggcggcacgacctactaccaggaggtcactcccctgctgagcagctgggggctgtccctggtgaccggttagtaatcgcatgttcatccgctcc

SEQ ID NO: 10 - protein, 361 aa, Artificial sequence
  Synthetic Construct
AFSSSIASAATVPAEPASEAQTMMEALQRDLGLTPLGAEELLSAQEEAIETDAEATEAAGASYGGSLFDTETLQLTVLVTDASAVEAVEATGAEATVVSHGAEGLAEVVDALDETGGREGVVGWYPDVESDTVVVQVAEGASADGLIEAAGVDPSAVRVEETSETPRLYADIVGGEAYYMGGGRCSVGFAVTDGSGAGGFVTAGHCGTVGTGAESSDGSGSGTFQESVFPGSDGAFVAATSNWNVTNLVSRYDSGSPQAVSGSSQAPEGSAVCRSGSTTGWHCGTIEARGQTVNYPQGTVQDLTRTDVCAEPGDSGGSFIAGSQAQGVTSGGSGNCTSGGTTYYQEVTPLLSSWGLSLVTG

SEQ ID NO: 11 - DNA, 44 bp, Artificial sequence
  Primer 1485
ggagcggatgaacatgcgattactaaccggtcaccagggacagc

SEQ ID NO: 12 - DNA, 44 bp, Artificial sequence; identical to SEQ ID NO: 11
  Primer 1424

SEQ ID NO: 13 - DNA, 1109 bp, Artificial sequence
  Fusion of Savinase signal with S2A protease
  CDS (1)..(1086)
  sig_peptide (1)..(21): Partial Savinase signal
  mat_peptide (520)..(1086)
agttcatcgatcgcatcggctgcgcccggccccgtcccccagacccccgtcgccgacgacagcgccgccagcatgaccgaagcgctcaagcgtgacctcaacctctcctcggccgaggccgaggagctgctctcggcgcaggaagccgcgatcgagaccgacgccgaggccgccgaggccgcgggagaggcctacggcggctccctgttcgacaccgaaaccctcgaactcaccgtgctggtgaccgacaccacggccgtcgacgcggtcgaggccaccggagccgaggccaccgtggtcacccacggcaccgacggcctggccgaggtcgtggaggacctcaacagcgccgacgccccggcgggcgtcctcggctggtaccccgacatggagagcgacaccgtggtggtcgaggtgctggagggctccgacgccgacgtcgccgccctgctcgccgacgccggcgtggacgcctccgccgtccgggtggaggaggcggaggaggtcccgcaggtctacgccaacatcatcggcggcctggcctacaccatgggcggacgctgctccgtcggcttcgcggcgaccaacagcgccggacagcccggtttcgtgacggcgggccactgcggcaccgtcggcaccgccgtgaccatcggcgacggccgcggcgtcttcgagcgctcggtcttccccggcaacgacgccgccttcgtccgcggcacctccaacttcaccctgaccaacctggtctcccgctacaactccggcggccaccaggcggtgaccggcaccagccaggccccggccggctcggccgtctgccgctccggctccaccaccggctggcactgcggcaccatccaggcccgcaaccagaccgtgcgctacccgcagggcaccgtcaacgcgctcacccgcaccaacgtgtgcgccgagcccggtgactccggcggctcgttcatctccggctcgcaggcccagggcgtcacctccggcggctccggcaactgctccttcggcggcacgacctactaccaggaggtcgccccgatgatcaactcctggggcgttcgcatccgcaccagctgatcgcatgttcaatccgctcc

SEQ ID NO: 14 - protein, 362 aa, Artificial sequence
  Synthetic Construct
SSSIASAAPGPVPQTPVADDSAASMTEALKRDLNLSSAEAEELLSAQEAAIETDAEAAEAAGEAYGGSLFDTETLELTVLVTDTTAVDAVEATGAEATVVTHGTDGLAEVVEDLNSADAPAGVLGWYPDMESDTVVVEVLEGSDADVAALLADAGVDASAVRVEEAEEVPQVYANIIGGLAYTMGGRCSVGFAATNSAGQPGFVTAGHCGTVGTAVTIGDGRGVFERSVFPGNDAAFVRGTSNFTLTNLVSRYNSGGHQAVTGTSQAPAGSAVCRSGSTTGWHCGTIQARNQTVRYPQGTVNALTRTNVCAEPGDSGGSFISGSQAQGVTSGGSGNCSFGGTTYYQEVAPMINSWGVRIRTS

SEQ ID NO: 15 - DNA, 42 bp, Artificial sequence
  Primer 1718
agttcatcgatcgcatcggctgcgcccggccccgtcccccag

SEQ ID NO: 16 - DNA, 41 bp, Artificial sequence
  Primer 1720
ggagcggattgaacatgcgatcagctggtgcggatgcgaac

SEQ ID NO: 17 - DNA, 1146 bp, Nocardiopsis dassonvillei subsp. dassonvillei DSM 43235
  CDS (1)..(1143)
  sig_peptide (1)..(87)
  mat_peptide (568)..(1143)
atgcgaccctcccccgctatctccgctatcggcaccggcgcactcgcgttcggtctggcgttctccgtgacgccgggcgccagtgcggcgaccgtaccggccgagccagcgagcgaggcccagacgatgatggaagcgctgcagagagacctcggcctcaccccgctcggggccgaggagctgctctcggcgcaggaagaggcgatcgagaccgacgccgaggccaccgaggccgcgggagcgtcctacggcggctccctgttcgacaccgagaccctccagctcaccgtgctggtgaccgacgcctcggccgtcgaggcggtggaggccaccggcgccgaggccaccgtggtctcacacggcgcagagggcctggccgaggtggtcgacgcgctcgacgagaccggcggccgggaaggggtcgtcggctggtacccggacgtggagagcgacaccgtcgtggtccaggtcgccgagggcgccagcgccgacggcctcatcgaggccgcgggcgtggacccctccgccgtccgggtggaggagaccagtgagactccgcgcctgtacgccgacatcgtcggcggcgaggcgtactacatgggcggcggacgctgctcggtcgggttcgccgtgaccgacggctccggcgcgggcggcttcgtgacggcgggccactgcggcaccgtcggcaccggcgccgagagctccgacggcagcggctccggaaccttccaggagtccgtcttcccgggcagcgacggcgccttcgtcgcggccacctccaactggaacgtgaccaacctggtcagccggtacgactccggcagcccccaggcggtgtcgggttccagccaggccccggagggctcggcggtgtgccgctccggctccaccaccggctggcactgcgggaccatcgaggcccgcggccagacggtgaactacccgcagggcacggtccaggacctgacccggacggacgtgtgcgccgagcccggtgactccggcggctcgttcatcgccggttcgcaggcccagggcgtcacctccggcggctcgggcaactgcacctccggcggcacgacctactaccaggaggtcactcccctgctgagcagctgggggctgtccctggtgaccggttag

SEQ ID NO: 18 - protein, 381 aa, Nocardiopsis dassonvillei subsp. dassonvillei DSM 43235
MRPSPAISAIGTGALAFGLAFSVTPGASAATVPAEPASEAQTMMEALQRDLGLTPLGAEELLSAQEEAIETDAEATEAAGASYGGSLFDTETLQLTVLVTDASAVEAVEATGAEATVVSHGAEGLAEVVDALDETGGREGVVGWYPDVESDTVVVQVAEGASADGLIEAAGVDPSAVRVEETSETPRLYADIVGGEAYYMGGGRCSVGFAVTDGSGAGGFVTAGHCGTVGTGAESSDGSGSGTFQESVFPGSDGAFVAATSNWNVTNLVSRYDSGSPQAVSGSSQAPEGSAVCRSGSTTGWHCGTIEARGQTVNYPQGTVQDLTRTDVCAEPGDSGGSFIAGSQAQGVTSGGSGNCTSGGTTYYQEVTPLLSSWGLSLVTG

SEQ ID NO: 19 - DNA, 1152 bp, Nocardiopsis prasina DSM 15649
  CDS (1)..(1149)
  sig_peptide (1)..(87)
  mat_peptide (574)..(1149)
atgcgaccctcccccgtcatctccgcgatcggcacgggagcactggccttcgggctcgcgctctcggtcgcgcccggcgcctccgccgtcaccgcacccaccgagcccgcgccccagggcgaggcggccaccatgcaggaagcgcttgagagggacttcggcctcaccccgttcgaggccgaagacctgctcgaagcccagaatgacgctctcgggatcgacacggcggcggccaaggccgccggtgacgcctacgcgggctccgtgttcgacaccgacaccctggaactgaccgtcctgctcacggacgccggagccgtgtcggacgtcgaggccaccggcgccgggaccgaactggtctcgtacggcaccgagggcctggcggagatcatggacgagctcgacgcagccggcgcccagccgggtgtcgtcggctggtacccggacctcgccggcgacaccgtcgtcatcgaggccaccgacacctccgaggcccagagcttcgtcgaggccgcgggcgtggactcctccgccgtccaggtggagcagaccgacgaggcgccgcagctgtacgccgacatcgtcggcggtgacgcctactacatgggcggcgggcgctgctcggtcggattcgcggtcaccgacagttccggcaacgacggattcgtgacggccggccactgcggcacggtcggcacctccgccgacagcgaggacggcagcggctccggtgtgttcgaggagtccatcttcccgggcaacgacgcggccttcgtcagttcgacgtccaactggaccgtcaccaacctggtcaacatgtacagctcgggtggcacccagtccgtcggcggctccagccaggccccggtcggcgcggccgtctgccgttccggctccaccacgggctggcactgcgggtccatcgaggcccgcgggcagtcggtgagctacccggagggcaccgtcaccgacatgacccgtaccgacgtgtgcgccgagcccggcgactccggcggttcgttcatcgccgacgaccaggcccagggcatgacctcgggcggctccggcaactgctcctccggtggtaccacgtactaccaggaggtcggcccggcgctgagcacctggaacctcagcctcgtcaccagctag

SEQ ID NO: 20 - protein, 383 aa, Nocardiopsis prasina DSM 15649
MRPSPVISAIGTGALAFGLALSVAPGASAVTAPTEPAPQGEAATMQEALERDFGLTPFEAEDLLEAQNDALGIDTAAAKAAGDAYAGSVFDTDTLELTVLLTDAGAVSDVEATGAGTELVSYGTEGLAEIMDELDAAGAQPGVVGWYPDLAGDTVVIEATDTSEAQSFVEAAGVDSSAVQVEQTDEAPQLYADIVGGDAYYMGGGRCSVGFAVTDSSGNDGFVTAGHCGTVGTSADSEDGSGSGVFEESIFPGNDAAFVSSTSNWTVTNLVNMYSSGGTQSVGGSSQAPVGAAVCRSGSTTGWHCGSIEARGQSVSYPEGTVTDMTRTDVCAEPGDSGGSFIADDQAQGMTSGGSGNCSSGGTTYYQEVGPALSTWNLSLVTS

SEQ ID NO: 21 - DNA, 1152 bp, Nocardiopsis prasina DSM 14010
  CDS (1)..(1149)
  sig_peptide (1)..(87)
  mat_peptide (574)..(1149)
atgcgaccctcccccgtcatctccgcgatcggcacgggagcgctggccttcgggctcgcgctctcggtcgctcccggcgcctccgccgtgaccgcccccgccgagccctcgccccagggcgaggcgaccaccatgcaggaagcgcttgagagggacttcggcctcaccccgttcgaggccgacgacctgctcgaagcccagaaggaggccctcgggatcgacacggcggcggccgaggccgccggcgacgcctacgcgggctccgtgttcgacaccgacaccctggaactgaccgtcctgctcacggacggcggcccggcgtcggacgtcgaggccgccggcgccgagacctcggtggtctcccacggcaccgacggcctggcggcgatcatggacgagctcgacgcggtcggcgcccagccgggtgtcgtcggctggtaccccgacctcgccagcgacacggtggtcgtcgaggccaccgacgcgtccgacgcccagggcttcatcgaggccgccggcgtggactcctccgccgtccaggtggaggagaccgacgagtcgcccgagctgtacgccgacatcgtcggcggcgacgcctactacatgggcggcggacgctgctcggtgggcttcgcggccaccgacagcgcgggcaacgacggattcgtgacggccggccactgcggcaccgtcggcacctccgccgacagcgaggacggcagcggctccggtgtgttcgaggagtcgatcttcccgggcaacgacgccgccttcgtccggtccacgtccaactggaccgtcaccaacctggtcaacatgtacagctccggcggcacccagtccgtcggcggctccacccaggccccggtcggcgcggccgtgtgccgctccggttccaccacgggctggcactgcggcaccatcgaggcccgaggccagtcggtgagctacccggagggcaccgtcaacgacatgacccggaccaacgtgtgcgccgagcccggcgactccggcggttcgttcatctccgacgaccaggcccagggcatgacctcgggcggctccggcaactgcacctccggtggtacgacgtactaccaggaggtcggcccggcgctgagcacctggaacctcagcctcgtcacgagctag

SEQ ID NO: 22 - protein, 383 aa, Nocardiopsis prasina DSM 14010
MRPSPVISAIGTGALAFGLALSVAPGASAVTAPAEPSPQGEATTMQEALERDFGLTPFEADDLLEAQKEALGIDTAAAEAAGDAYAGSVFDTDTLELTVLLTDGGPASDVEAAGAETSVVSHGTDGLAAIMDELDAVGAQPGVVGWYPDLASDTVVVEATDASDAQGFIEAAGVDSSAVQVEETDESPELYADIVGGDAYYMGGGRCSVGFAATDSAGNDGFVTAGHCGTVGTSADSEDGSGSGVFEESIFPGNDAAFVRSTSNWTVTNLVNMYSSGGTQSVGGSTQAPVGAAVCRSGSTTGWHCGTIEARGQSVSYPEGTVNDMTRTNVCAEPGDSGGSFISDDQAQGMTSGGSGNCTSGGTTYYQEVGPALSTWNLSLVTS

SEQ ID NO: 23 - DNA, 1155 bp, Nocardiopsis sp. DSM 16424
  CDS (1)..(1152)
  sig_peptide (1)..(87)
  mat_peptide (586)..(1152)
atgagaccctccaccatcgcctccgccgtcggcacaggagcactggccttcggtctggcactgtccatggcccccggagccctcgcggcgcccggccccgtcccccagacccccgtcgccgacgacagcgccgccagcatgaccgaagcgctcaagcgtgacctcaacctctcctcggccgaggccgaggagctgctctcggcgcaggaagccgcgatcgagaccgacgccgaggccgccgaggccgcgggagaggcctacggcggctccctgttcgacaccgaaaccctcgaactcaccgtgctggtgaccgacaccacggccgtcgacgcggtcgaggccaccggagccgaggccaccgtggtcacccacggcaccgacggcctggccgaggtcgtggaggacctcaacagcgccgacgccccggcgggcgtcctcggctggtaccccgacatggagagcgacaccgtggtggtcgaggtgctggagggctccgacgccgacgtcgccgccctgctcgccgacgccggcgtggacgcctccgccgtccgggtggaggaggcggaggaggtcccgcaggtctacgccaacatcatcggcggcctggcctacaccatgggcggacgctgctccgtcggcttcgcggcgaccaacagcgccggacagcccggtttcgtgacggcgggccactgcggcaccgtcggcaccgccgtgaccatcggcgacggccgcggcgtcttcgagcgctcggtcttccccggcaacgacgccgccttcgtccgcggcacctccaacttcaccctgaccaacctggtctcccgctacaactccggcggccaccaggcggtgaccggcaccagccaggccccggccggctcggccgtctgccgctccggctccaccaccggctggcactgcggcaccatccaggcccgcaaccagaccgtgcgctacccgcagggcaccgtcaacgcgctcacccgcaccaacgtgtgcgccgagcccggtgactccggcggctcgttcatctccggctcgcaggcccagggcgtcacctccggcggctccggcaactgctccttcggcggcacgacctactaccaggaggtcgccccgatgatcaactcctggggcgttcgcatccgcaccagctga

SEQ ID NO: 24 - protein, 384 aa, Nocardiopsis sp. DSM 16424
MRPSTIASAVGTGALAFGLALSMAPGALAAPGPVPQTPVADDSAASMTEALKRDLNLSSAEAEELLSAQEAAIETDAEAAEAAGEAYGGSLFDTETLELTVLVTDTTAVDAVEATGAEATVVTHGTDGLAEVVEDLNSADAPAGVLGWYPDMESDTVVVEVLEGSDADVAALLADAGVDASAVRVEEAEEVPQVYANIIGGLAYTMGGRCSVGFAATNSAGQPGFVTAGHCGTVGTAVTIGDGRGVFERSVFPGNDAAFVRGTSNFTLTNLVSRYNSGGHQAVTGTSQAPAGSAVCRSGSTTGWHCGTIQARNQTVRYPQGTVNALTRTNVCAEPGDSGGSFISGSQAQGVTSGGSGNCSFGGTTYYQEVAPMINSWGVRIRTS

SEQ ID NO: 25 - DNA, 1152 bp, Nocardiopsis alkaliphila DSM 44657
  CDS (1)..(1149)
  sig_peptide (1)..(87)
  mat_peptide (586)..(1149)
atgcgaccctcccccgttgtctccgccataggtacaggagccttggccttcggcctggctctgggcacttcccccgcggccatcgccgcccccgccccccagtcccccgacaccgaaacgcaggccgaggccgtcaccatggccgaagccctccaacgcgatctcggtctgtcctcctccgaggccaccgaactcctcgccgcacaggccgaggcgttcgaggtcgacgaggccgccaccgaggccgccgccgacgcctacggcggctccctcttcgacaccgacagcctcgaactgaccgtgctggtcaccgacagcgccgccgtcgacgcggtcgaggccaccggcgccaaggccgaggtcgtcgaccacggtatcgagggcctcgaggagatcgtcgacgaactcaacgagtccaacgccaagtcgggcgtcgtcggttggtaccccgacgtggccggtgacacggtcgtcctggaggtcatggaaggctccgaggccgacgtggacgccctgctcgccgagaccggggtcgacgccgccgacgtcacggtggagaccaccaccgagcagcccgagctctacgccgacatcatcggtggcctggcctacaccatgggcggacgttgctcggtcggcttcgccgccaccaactcctccggccagcccggattcgtcaccgccggccactgcggcagtgtcggcaccggcgtcaccatcggtaacggccggggcgtcttcgagcgttccatcttcccgggcaacgacgccgccttcgtccgtggcacgtccaacttcaccctgaccaacctggtcagccgctacaactccggcggctacgccacggtgtccgggtcctccgcggccccgatcggctcccaggtgtgccgctccggctccaccaccggctggcactgcggcaccatccaggcccgcaaccagacggtgcgctacccgcagggcaccgtccaggccctgacccgcaccagcgtgtgcgccgagcccggtgactccggtggttccttcatctccggcagccaggcccagggcgtcacctccggtggctcgggcaactgccgcaccggtggcacgacctactaccaggaggtcaaccccatgctcaacagctggggcctgcgtctgcgcacctga

SEQ ID NO: 26 - protein, 383 aa, Nocardiopsis alkaliphila DSM 44657
MRPSPVVSAIGTGALAFGLALGTSPAAIAAPAPQSPDTETQAEAVTMAEALQRDLGLSSSEATELLAAQAEAFEVDEAATEAAADAYGGSLFDTDSLELTVLVTDSAAVDAVEATGAKAEVVDHGIEGLEEIVDELNESNAKSGVVGWYPDVAGDTVVLEVMEGSEADVDALLAETGVDAADVTVETTTEQPELYADIIGGLAYTMGGRCSVGFAATNSSGQPGFVTAGHCGSVGTGVTIGNGRGVFERSIFPGNDAAFVRGTSNFTLTNLVSRYNSGGYATVSGSSAAPIGSQVCRSGSTTGWHCGTIQARNQTVRYPQGTVQALTRTSVCAEPGDSGGSFISGSQAQGVTSGGSGNCRTGGTTYYQEVNPMLNSWGLRLRT

SEQ ID NO: 27 - DNA, 1155 bp, Nocardiopsis lucentis DSM 44048
  CDS (1)..(1152)
  sig_peptide (1)..(87)
  mat_peptide (586)..(1152)
atgcgaccctcccccgttatctccgccctaggaaccggcgccctcgccttcggactggtcatcaccatggccccgggcgtgaacgccggaaccgtacccaccccccaggcccccgtccccgacgacgaggccaccaccatgctcgaagccatggagagggatctcgacctcaccccgttcgaggccgaggaactcttcgaggcacaggaagaggccatcgacctcgacgaggaggccaccgaagcggccggtgcggcctacggcggttcgctcttcgacaccgaaacccacgaactcaccgtcctggtgaccgacgtcgacgcggtcgaggccgtggaggccaccggagccgccgccgaggtcgtctcccacggctccgacggtctggccgacatcgtcgaggacctcaacgccaccgacgccggcagcgaggtcgtgggctggtaccccgacgtcaccagcgacagcgtggtcgtcgaggtggtcgagggctccgacgtcgacgtcgactccatcgtcgagggcacgggcgtcgacccggcggtcatcgaggtccaggaggtctccgaacagcctcagacctacgccaacatcatcggcggcctggcctactacatgagctcgggcggccgctgctcggtcggcttccccgccaccaacagctccggccagccgggcttcgtcacggcgggccactgcggcaccgtcggcaccggcgtcaccatcggcaacggccgcggcaccttcgagcgctccgtgttccccggcaacgacgccgccttcgtccgaggcacgtccaacttcacgctgtacaacctcgtctaccgctacagcggctaccagaccgtgacgggcagcaacgccgccccgatcggctcgtccatctgccgttccggttccaccaccggctggcactgcggcaccatccaggcccgcaaccagaccgtccggtacccgcagggcaccgtctactacctgacccgtaccaacgtgtgcgccgagcccggcgactccggaggctccttcatctccggaacgcaggcccagggcatgacctccggcggctccggcaactgcagcagcggtggcaccaccttctaccaggaggtggacccggtggagagcgcctggggcgtgcgactgcgcaccagctag

SEQ ID NO: 28 - protein, 384 aa, Nocardiopsis lucentis DSM 44048
MRPSPVISALGTGALAFGLVITMAPGVNAGTVPTPQAPVPDDEATTMLEAMERDLDLTPFEAEELFEAQEEAIDLDEEATEAAGAAYGGSLFDTETHELTVLVTDVDAVEAVEATGAAAEVVSHGSDGLADIVEDLNATDAGSEVVGWYPDVTSDSVVVEVVEGSDVDVDSIVEGTGVDPAVIEVQEVSEQPQTYANIIGGLAYYMSSGGRCSVGFPATNSSGQPGFVTAGHCGTVGTGVTIGNGRGTFERSVFPGNDAAFVRGTSNFTLYNLVYRYSGYQTVTGSNAAPIGSSICRSGSTTGWHCGTIQARNQTVRYPQGTVYYLTRTNVCAEPGDSGGSFISGTQAQGMTSGGSGNCSSGGTTFYQEVDPVESAWGVRLRTS

SEQ ID NO: 29 - DNA, 1068 bp, Nocardiopsis alba DSM 15647
  CDS (1)..(1065)
  mat_peptide (502)..(1065)
gcgaccggccccctcccccagtcccccaccccggatgaagccgaggccaccaccatggtcgaggccctccagcgcgacctcggcctgtccccctctcaggccgacgagctcctcgaggcgcaggccgagtccttcgagatcgacgaggccgccaccgcggccgcagccgactcctacggcggctccatcttcgacaccgacagcctcaccctgaccgtcctggtcaccgacgcctccgccgtcgaggcggtcgaggccgccggcgccgaggccaaggtggtctcgcacggcatggagggcctggaggagatcgtcgccgacctgaacgcggccgacgctcagcccggcgtcgtgggctggtaccccgacatccactccgacacggtcgtcctcgaggtcctcgagggctccggtgccgacgtggactccctgctcgccgacgccggtgtggacaccgccgacgtcaaggtggagagcaccaccgagcagcccgagctgtacgccgacatcatcggcggtctcgcctacaccatgggtgggcgctgctcggtcggcttcgcggccaccaacgcctccggccagcccgggttcgtcaccgccggccactgcggcaccgtcggcaccccggtcagcatcggcaacggccagggcgtcttcgagcgttccgtcttccccggcaacgactccgccttcgtccgcggcacctcgaacttcaccctgaccaacctggtcagccgctacaacaccggtggttacgcgaccgtctccggctcctcgcaggcggcgatcggctcgcagatctgccgttccggctccaccaccggctggcactgcggcaccgtccaggcccgcggccagacggtgagctacccccagggcaccgtgcagaacctgacccgcaccaacgtctgcgccgagcccggtgactccggcggctccttcatctccggcagccaggcccagggcgtcacctccggtggctccggcaactgctccttcggtggcaccacctactaccaggaggtcaacccgatgctgagcagctggggtctgaccctgcgcacctga

SEQ ID NO: 30 - protein, 355 aa, Nocardiopsis alba DSM 15647
ATGPLPQSPTPDEAEATTMVEALQRDLGLSPSQADELLEAQAESFEIDEAATAAAADSYGGSIFDTDSLTLTVLVTDASAVEAVEAAGAEAKVVSHGMEGLEEIVADLNAADAQPGVVGWYPDIHSDTVVLEVLEGSGADVDSLLADAGVDTADVKVESTTEQPELYADIIGGLAYTMGGRCSVGFAATNASGQPGFVTAGHCGTVGTPVSIGNGQGVFERSVFPGNDSAFVRGTSNFTLTNLVSRYNTGGYATVSGSSQAAIGSQICRSGSTTGWHCGTVQARGQTVSYPQGTVQNLTRTNVCAEPGDSGGSFISGSQAQGVTSGGSGNCSFGGTTYYQEVNPMLSSWGLTLRT

SEQ ID NO: 31 - DNA, 1062 bp, Nocardiopsis prasina DSM 15648
  CDS (1)..(1059)
  mat_peptide (496)..(1059)
gccaccggaccgctcccccagtcacccaccccggaggccgacgccgtctccatgcaggaggcgctccagcgcgacctcggcctgaccccgcttgaggccgatgaactgctggccgcccaggacaccgccttcgaggtcgacgaggccgcggccgcggccgccggggacgcctacggcggctccgtcttcgacaccgagaccctggaactgaccgtcctggtcaccgacgccgcctcggtcgaggctgtggaggccaccggcgcgggtaccgaactcgtctcctacggcatcgagggcctcgacgagatcatccaggatctcaacgccgccgacgccgtccccggcgtggtcggctggtacccggacgtggcgggtgacaccgtcgtcctggaggtcctggagggttccggagccgacgtgagcggcctgctcgccgacgccggcgtggacgcctcggccgtcgaggtgaccagcagtgcgcagcccgagctctacgccgacatcatcggcggtctggcctacaccatgggcggccgctgttcggtcggattcgcggccaccaacgccgccggtcagcccggattcgtcaccgccggtcactgtggccgcgtgggcacccaggtgagcatcggcaacggccagggcgtcttcgagcagtccatcttcccgggcaacgacgccgccttcgtccgcggcacgtccaacttcacgctgaccaacctggtcagccgctacaacaccggcggttacgccaccgtcgccggccacaaccaggcgcccatcggctcctccgtctgccgctccggctccaccaccggctggcactgcggcaccatccaggcccgcggccagtcggtgagctaccccgagggcaccgtcaccaacatgacccggaccaccgtgtgcgccgagcccggcgactccggcggctcctacatctccggcaaccaggcccagggcgtcacctccggcggctccggcaactgccgcaccggcgggaccaccttctaccaggaggtcacccccatggtgaactcctggggcgtccgtctccggacctaa

SEQ ID NO: 32 - protein, 353 aa, Nocardiopsis prasina DSM 15648
ATGPLPQSPTPEADAVSMQEALQRDLGLTPLEADELLAAQDTAFEVDEAAAAAAGDAYGGSVFDTETLELTVLVTDAASVEAVEATGAGTELVSYGIEGLDEIIQDLNAADAVPGVVGWYPDVAGDTVVLEVLEGSGADVSGLLADAGVDASAVEVTSSAQPELYADIIGGLAYTMGGRCSVGFAATNAAGQPGFVTAGHCGRVGTQVSIGNGQGVFEQSIFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGNQAQGVTSGGSGNCRTGGTTFYQEVTPMVNSWGVRLRT

SEQ ID NO: 33 - DNA, 1065 bp, Nocardiopsis dassonvillei subsp. dassonvillei DSM 43235
  CDS (1)..(1062)
  mat_peptide (499)..(1062)
gctccggcccccgtcccccagacccccgtcgccgacgacagcgccgccagcatgaccgaggcgctcaagcgcgacctcgacctcacctcggccgaggccgaggagcttctctcggcgcaggaagccgccatcgagaccgacgccgaggccaccgaggccgcgggcgaggcctacggcggctcactgttcgacaccgagaccctcgaactcaccgtgctggtcaccgacgcctccgccgtcgaggcggtcgaggccaccggagcccaggccaccgtcgtctcccacggcaccgagggcctgaccgaggtcgtggaggacctcaacggcgccgaggttcccgagagcgtcctcggctggtacccggacgtggagagcgacaccgtcgtggtcgaggtgctggagggctccgacgccgacgtcgccgccctgctcgccgacgccggtgtggactcctcctcggtccgggtggaggaggccgaggaggccccgcaggtctacgccgacatcatcggcggcctggcctactacatgggcggccgctgctccgtcggcttcgccgcgaccaacagcgccggtcagcccggtttcgtcaccgccggccactgcggcaccgtcggcaccggcgtgaccatcggcaacggcaccggcaccttccagaactcggtcttccccggcaacgacgccgccttcgtccgcggcacctccaacttcaccctgaccaacctggtctcgcgctacaactccggcggctaccagtcggtgaccggtaccagccaggccccggccggctcggccgtgtgccgctccggctccaccaccggctggcactgcggcaccatccaggcccgcaaccagaccgtgcgctacccgcagggcaccgtctactcgctcacccgcaccaacgtgtgcgccgagcccggcgactccggcggttcgttcatctccggctcgcaggcccagggcgtcacctccggcggctccggcaactgctccgtcggcggcacgacctactaccaggaggtcaccccgatgatcaactcctggggtgtcaggatccggacctaa

SEQ ID NO: 34 - protein, 354 aa, Nocardiopsis dassonvillei subsp. dassonvillei DSM 43235
APAPVPQTPVADDSAASMTEALKRDLDLTSAEAEELLSAQEAAIETDAEATEAAGEAYGGSLFDTETLELTVLVTDASAVEAVEATGAQATVVSHGTEGLTEVVEDLNGAEVPESVLGWYPDVESDTVVVEVLEGSDADVAALLADAGVDSSSVRVEEAEEAPQVYADIIGGLAYYMGGRCSVGFAATNSAGQPGFVTAGHCGTVGTGVTIGNGTGTFQNSVFPGNDAAFVRGTSNFTLTNLVSRYNSGGYQSVTGTSQAPAGSAVCRSGSTTGWHCGTIQARNQTVRYPQGTVYSLTRTNVCAEPGDSGGSFISGSQAQGVTSGGSGNCSVGGTTYYQEVTPMINSWGVRIRT

SEQ ID NO: 35 - DNA, 1143 bp, Artificial sequence
  Improved Synthetic protease gene
  CDS (1)..(1140): Protease
  sig_peptide (1)..(81)
  mat_peptide (577)..(1140)
atgaaaaaaccgctgggaaaaattgtcgcaagcacagcacttcttatttcagtggcatttagctcatctattgcatcagctgctacgggagctttaccgcagtctccgacaccggaagcagatgcagtgtcaatgcaagaagcactgcaaagagatcttgatcttacatcagcagaagcagaagaacttcttgctgcacaagatacagcatttgaagtggatgaagcagcggcagaagcagcaggagatgcatatggcggctcagtttttgatacagaatcacttgaacttacggttcttgttacagatgcagcagcagttgaagcagttgaagcaacaggtgcaggaacagaacttgtttcatatggaattgatggccttgatgaaattgttcaagaactgaatgcagctgatgctgttccgggcgttgtcggctggtatccggatgttgctggagatacagttgtccttgaagttcttgaaggatcaggcgcagatgtttcaggcctgctggcagatgcaggagtcgatgcatcagcagttgaagttacaacatcagatcaaccggaactttatgcagatattattggcggcctggcatatacaatgggcggcagatgcagcgttggctttgcagcaacaaatgcagcaggccaaccgggctttgttacagcaggccattgcggcagagttggcacacaggttacaattggcaatggcagaggcgtttttgaacaaagcgtttttccgggcaatgatgcagcatttgttagaggcacatcaaattttacacttacaaatctggtttcaagatataatacaggcggctatgcaacagttgcaggccataatcaagcaccgattggctcatcagtttgcagatcaggctcaacaacaggctggcattgcggcacaattcaagcaagaggccaaagcgttagctatccggaaggcacagttacaaatatgacaagaacaacagtttgtgcagaaccgggcgattcaggcggctcatatattagcggcacacaagcacaaggcgttacatcaggcggctcaggcaattgcagaacaggcggcacaacattttaccaagaagttacaccgatggttaattcatggggcgttagacttagaacataa

SEQ ID NO: 36 - protein, 380 aa, Artificial sequence; identical to SEQ ID NO: 4
  Synthetic Construct

SEQ ID NO: 37 - DNA, 1143 bp, Artificial sequence
  Optimized Synthetic protease gene
  CDS (1)..(1140): Protease
  sig_peptide (1)..(81)
  mat_peptide (577)..(1140)
atgaaaaaaccgctgggaaaaattgtcgcaagcacagcacttcttatttcagtggcatttagctccagcattgcatcagctgctacgggagctttaccgcagtctccgacaccggaagcagatgcagtgtcaatgcaagaagcactgcaaagagatcttgatcttacatcagcagaagcagaagaacttcttgctgcacaagatacagcatttgaagtggatgaagcagcggcagaagcagcaggagatgcatatggcggctcagtttttgatacagaatcacttgaacttacggttcttgttacagatgcagcagcagttgaagcagttgaagcaacaggtgcaggaacagaacttgtttcatatggaattgatggccttgatgaaattgttcaagaactgaatgcagctgatgctgttccgggcgttgtcggctggtatccggatgttgctggagatacagttgtccttgaagttcttgaaggatcaggcgcagatgtttcaggcctgctggcagatgcaggagtcgatgcatcagcagttgaagttacaacatcagatcaaccggaactttatgcagatattattggcggcctggcatatacaatgggcggcagatgcagcgttggctttgcagcaacaaatgcagcaggccaaccgggctttgttacagcaggccattgcggcagagttggcacacaggttacaattggcaatggcagaggcgtttttgaacaaagcgtttttccgggcaatgatgcagcatttgttagaggcacatcaaattttacacttacaaatctggtttcaagatataatacaggcggctatgcaacagttgcaggccataatcaagcaccgattggctcatcagtttgcagatcaggctcaacaacaggctggcattgcggcacaattcaagcaagaggccaaagcgttagctatccggaaggcacagttacaaatatgacaagaacaacagtttgtgcagaaccgggcgattcaggcggctcatatattagcggcacacaagcacaaggcgttacatcaggcggctcaggcaattgcagaacaggcggcacaacattttaccaagaagttacaccgatggttaattcatggggcgtgcgccttcgcacataa

SEQ ID NO: 38 - protein, 380 aa, Artificial sequence; identical to SEQ ID NO: 4
  Synthetic Construct

SEQ ID NO: 39 - DNA, 1143 bp, Artificial sequence
  Optimized Synthetic protease gene
  CDS (1)..(1140): Protease
  sig_peptide (1)..(81)
  mat_peptide (577)..(1140)
atgaaaaaaccgctgggaaaaattgtcgcaagcacagcacttcttatttcagtggcatttagctcatctattgcatcagcagctacaggagcattaccgcagtctccgacaccggaagcagatgcagtctcaatgcaagaagcactgcaaagagatcttgatcttacatcagcagaagcagaagaacttcttgctgcacaagatacagcatttgaagtggatgaagcagcggcagaagcagcaggagatgcatatggcggctcagtttttgatacagaatcacttgaacttacggttcttgttacagatgcagcagcagttgaagcagttgaagcaacaggagcaggaacagaacttgtttcatatggaattgatggccttgatgaaattgttcaagaactgaatgcagctgatgctgttccgggcgttgttggctggtatccggatgttgctggagatacagttgtccttgaagttcttgaaggatcaggcgcagatgtttcaggcctgctggcagatgcaggagtcgatgcatcagcagttgaagttacaacatcagatcaaccggaactttatgcagatattattggcggcctggcatatacaatgggcggcagatgcagcgttggctttgctgcaacaaatgcagcaggccaaccgggctttgttacagcaggccattgcggcagagttggcacacaggttacaattggcaatggcagaggcgtttttgaacaaagcgtttttccgggcaatgatgcagcatttgttagaggcacatcaaattttacacttacaaacctggtttcaagatataatacaggcggctatgcaacagttgcaggccataatcaagcaccgattggctcatcagtttgcagatcaggctcaacaacaggctggcattgcggcacaattcaagcaagaggccaaagcgttagctatccggaaggcacagttacaaatatgacaagaacaacagtctgtgccgaaccgggcgattcaggcggctcatatattagcggcacgcaggcgcaaggcgttacatcaggcggctcaggcaattgcagaacaggcggcactacattttaccaagaagttacaccgatggtaaattcatggggcgtgcgccttcgcacataa

SEQ ID NO: 40 - protein, 380 aa, Artificial sequence; identical to SEQ ID NO: 4
  Synthetic Construct